(19) United States
(12) Patent Application Publication
(10) Pub. No.: US 2012/0053184 A1
(43) Pub. Date: Mar. 1, 2012

(54) SALTS OF 3-(1H-IND0L-3-YL)-4-[2-(4-METHYL-PIPERAZIN-I-YL)-QUINAZOLIN-4-YL]-PYRROLE-2, 5-DI ONE

(76) Inventors: Piotr Karpinski, Lincoln Park, NJ (US); Dimitris Papoutsakis, Acton, MA (US); Guy Yowell, Boonton, NJ (US)

(21) Appl. No.: 13/291,705

(22) Filed: Nov. 8, 2011

Related U.S. Application Data
(63) Continuation of application No. 12/529,943, filed on Sep. 4, 2009, now abandoned.
(60) Provisional application No. 60/893,954, filed on Mar. 9, 2007.

(51) Int. Cl. A61K 31/496; A61P 37/06; A61P 3/10; A61P 37/00; A61P 37/02; A61P 35/00; C07D 401/14; A61P 29/00

(52) U.S. Cl. 514/252.17; 544/284

(57) ABSTRACT

The invention provides acid addition salts of 3-(1.H.-indol-3-yl)-4-[2-(4-methyl-piperazin-1-yl)-quinazolin-4-yl]-pyrrole-2,5-dione, crystalline forms thereof, processes for the preparation thereof, pharmaceutical compositions comprising them and uses thereof in therapeutic treatment.

The present invention relates to acid addition salts of 3-(1.H.-indol-3-yl)-4-[2-(4-methyl-piperazin-1-yl)-quinazolin-4-yl]-pyrrole-2,5-dione, and crystalline forms thereof. Also provided are processes for the preparation thereof, pharmaceutical compositions comprising the compounds of the present invention and uses thereof in therapeutic treatment of warm-blooded animals, especially humans.

3-(1.H.-indol-3-yl)-4-[2-(4-methyl-piperazin-1-yl)-quinazolin-4-yl]-pyrrole-2,5-dione, hereinafter named “the compound of the invention”, can be represented by the following formula (I):

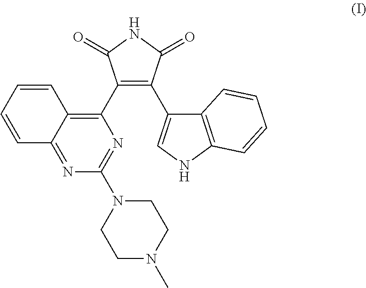

and is known from EP1337527, the entire disclosure of which is incorporated by reference, and can be synthesized as described therein.

The present invention relates to novel and improved salts, polymorphs and solvates thereof, of the known compound of formula (I). The compounds of the formula (I) include racemic or enantiomeric forms.

The free base of the compound of the invention shows relatively low solubility in aqueous media. Therefore it is not straightforward or easy to formulate it into pharmaceutical compositions, e.g. for oral administration.

In order to obtain a salt which can be formulated into pharmaceutical compositions, it is important the salt be stable in the solid state.

In accordance with the present invention it has now surprisingly been found that difficulties in formulating the free base can be overcome with the compounds of the present invention. It has been found that, unexpectedly, some salts of the compound of formula I, e.g. with specific acid, possess particularly beneficial pharmacokinetic properties and have further been found to possess a unique combination of favorable formulation properties which make them particularly suitable for the preparation of pharmaceutical compositions containing the compound of the invention adapted for oral administration.

The present invention include e.g. the following salts of the known compound of formula (I): benzoate, chloride, citrate, fumarate, lactate, maleate, malate, malonate, mesylate (e.g. methansulfonate), phosphate, succinate, and tartarate salts of 3-(1.H.-indol-3-yl)-4-[2-(4-methyl-piperazin-1-yl)-quinazolin-4-yl]-pyrrole-2,5-dione, preferably methansulfonate and malonate salts of 3-(1.H.-indol-3-yl)-4-[2-(4-methyl-piperazin-1-yl)-quinazolin-4-yl]pyrrole-2,5-dione (hereinafter named “salts of the invention”). Preferred salts are maleate salt and mesylate salt of 3-(1.H.-indol-3-yl)-4-[2-(4-methyl-piperazin-1-yl)-quinazolin-4-yl]-pyrrole-2,5-dione.

The salts of the invention, for example maleate and mesylate salt of 3-(1.H.-indol-3-yl)-4-[2-(4-methyl-piperazin-1-yl)-quinazolin-4-]-pyrrole-2,5-dione, exhibit very good stability in the solid sate, e.g. after one week at 80° C., in a close container, or at 80° C./75% r.h., in open container, or upon exposure to light (1200 kLux, 300-800 nm).

Such crystalline forms show improved stability and purity and thus e.g. easier handling in plant.

Furthermore, it has been surprisingly found in accordance with the present invention that under certain conditions crystalline forms or solvate forms can be obtained from the salts of the invention, for example from the salts obtained with benzoic acid, hydrochloric acid, citric acid, fumaric acid, maleic acid, malic acid, malonic acid, methanesulfonic acid, succinic acid or tartaric acid.

The crystalline forms of the salts of the invention, e.g. mesylate salt or maleate salt of 3-(1.H.-indol-3-yl)-4-[2-(4-methyl-piperazin-1-yl)-quinazolin-4-yl]-pyrrole-2,5-dione, are preferably essentially pure, e.g. in essentially pure form.

The term essentially pure in accordance with the present invention is means that the sum of related substances is less than 1%, preferably less than 0.75%, more preferably less than 0.5% and that the residual solvents and water are less than 1%, preferably less than 0.75%, more preferably less than 0.5% and still more preferably less than 0.25% by weight.

Criteria for selecting the salts of the invention include i) dissolution in water, with measurement of degradation products, and decoloration, ii) stability against heat in solid state, iii) exposure to light (e.g. Xenon light), iv) corosivity to steel.

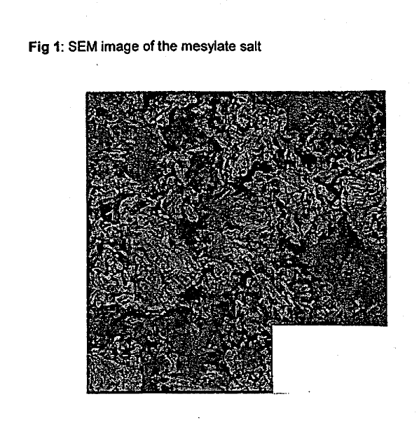

FIG. 1 shows the SEM image of the mesylate salt of 3-(1.H.-indol-3-yl)-4-[2-(4-methyl-piperazin-1-yl)-quinazolin-4-yl]-pyrrole-2,5-dione.

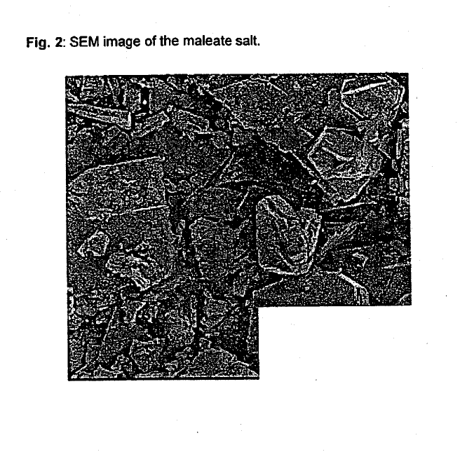

FIG. 2 shows the SEM image of the maleate salt of 3-(1.H.-indol-3-yl)-4-[2-(4-methyl-piperazin-1-yl)-quinazolin-4-yl]-pyrrole-2,5-dione.

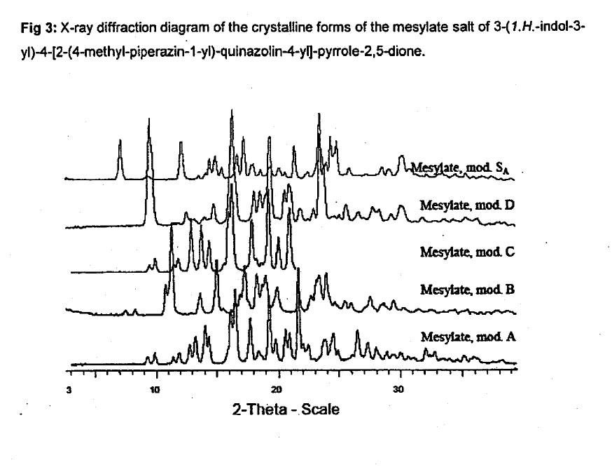

FIG. 3 shows the X-ray diffraction diagram of the crystalline forms of the mesylate salt of 3-(1.H.-indol-3-yl)-4-[2-(4-methyl-piperazin-1-yl)-quinazolin-4-yl]-pyrrole-2,5-dione. In the X-ray diagram, the angle of diffraction 2 theta is plotted on the horizontal axis (x-axis) and the peak intensity on the vertical (y-axis). X-ray powder diffraction patterns are measured on a Bruker D8 Discover diffractometer with Cu Kα radiation source (Kα1 radiation, wavelength λ=1.54056 Angström).

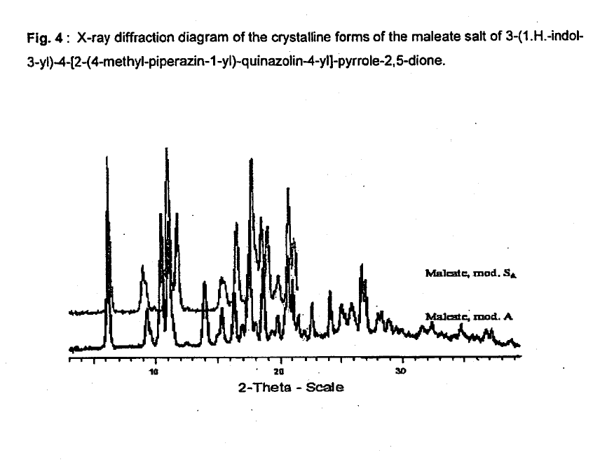

FIG. 4 shows the X-ray diffraction diagram of the crystalline forms of the maleate salt of 3-(1.H.-indol-3-yl)-4-[2-(4-methyl-piperazin-1-yl)-quinazolin-4-yl]-pyrrole-2,5-dione. In the X-ray diagram, the angle of diffraction 2 theta is plotted on the horizontal axis (x-axis) and the peak intensity on the vertical (y-axis). X-ray powder diffraction patterns are measured on a Bruker D8 Discover diffractometer with Cu Kα radiation source (Kα1 radiation, wavelength λ=1.54056 Angström).

In accordance with the present invention, the observed angle of diffraction 2 theta can deviate ±0.1°, ±0.2°, ±0.3°, preferably up to ±10% or ±20% of the above angles of refraction.

The salts of the invention may be prepared by suspending free base of 3-(1.H.-indol-3-yl)-4-[2-(4-methyl-piperazin-1-yl)-quinazolin-4-yl]-pyrrole-2,5-dione in an appropriate solvent, such as for example acetone, 2-propanol, ethanol, ethyl acetate, acetonitrile, or mixture thereof. A salt forming agent (SFA) is subsequently dissolved in the corresponding solvent (for tartaric, fumaric and citric acids some water may also be added to aid the solubilization of the SFA) and added to the suspension/solution of the free base. The mixture is stirred at a temperature comprised between 20° C. and 60° C., for example between 30° C. and 50° C., between 40° C. and 50° C. Most preferably the mixture is stirred either at ambient temperature.

The salts of the invention may be isolated by filtration and characterized e.g. by XRPD, thermal analysis and NMR spectroscopy.

The following salts were isolated as crystalline solids, which melted upon decomposition at temperatures exceeding 130° C.:

Benzoate: 1:1 salt; form (A), 154.2° C.; acetone solvate (134.8° C.).

Chloride: 1:1 salt; at least four distinct polymorphic forms (B; C; D; E); two isostructural solvates (S_{A} with acetone, 270.6° C. ; S_{B} with 2-propanol).

Citrate: 2:1 salt; one polymorphic form (A).

Fumarate: 2:1 salt; one polymorphic form (A), 162.0° C.; one methanol solvate (S_{A}).

Maleate: 1:1 salt; one polymorphic form (A), 180.2° C.

Malate: : 2:1 salt; polymorphic forms (A, 157.7° C.; B, 132.0° C.; C; D; E); solvates (S_{A} with methanol).

Malonate: 2:1 salt; isomorphic solvates, 174.6° C.

Methyl sulfonate: 1:1 salt; two polymorphs (A, 284.8° C.; B), one acetone solvate (S_{A}).

Succinate: 2:1 salt; one polymorphic form (A, 154.7° C.) isomorphic to fumarate salt, solvates.

Tartarate: 2:1 salt, one polymorphic form (A).

The values in ° C. indicate the melting points (temperature at which the salts melt with decomposition). The Melting points (in ° C.) are taken at a heating rate of 10 C/min.

Therefore the present invention provides:

1.1 A salt of 3-(1.H.-indol-3-yl)-4-[2-(4-methyl-piperazin-1-yl)-quinazolin-4-yl]pyrrole-2,5-dione formed with an acid selected from the group consisting of benzoic, hydrochloric, citric, fumaric, lactic, maleic, malic, malonic, methanesulfonic, phosphoric, succinic, and tartaric acid, preferably maleic or methanesulfonic acid.

1.2 A salt of 3-(1.H.-indol-3-yl)-4-[2-(4-methyl-piperazin-1-yl)-quinazolin-4-yl]-pyrrole-2,5-dione in crystalline or solvate form wherein the salt is formed with an acid selected from the group consisting of benzoic, hydrochloric, citric, fumaric, maleic, malic, malonic, methanesulfonic, succinic and tartaric acid, preferably maleic or methanesulfonic acid.

1.3 A salt of 3-(1.H.-indol-3-yl)-4-[2-(4-methyl-piperazin-1-yl)-quinazolin-4-yl]-pyrrole-2,5-dione or crystalline form thereof as indicated under 1.1. or 1.2 above which is essentially pure, e.g. in essentially pure form.

The invention also includes a process for the preparation of the salts of the invention which comprises reacting the compound of formula I in free base form with an appropriate acid and recovering from the reaction mixture the resultant salt. The process of the invention may be effected in conventional manner, e. g. by reaction in an appropriate inert solvent such as acetone, acetonitrile, ethyl acetate, ethanol, 2-propoanol, or t-butyl methyl ether. The mixture can be strirred e.g. at ambient temperature, at a temperature comprised between 40 and 50° C. or can be heated.

Optionally a salt forming agent may be added, e.g. subsequently dissolved in the solvent and added to the suspension/solution of the free base. Some water may be added in order to aid the solubilization of the salt forming agent.

In accordance with the present invention a process for the crystallization of the salts of the invention is provided. The precise conditions under which crystals are formed may now be empirically determined and a number of methods are suitable in practice, including the crystallization conditions as described in Examples.

The salts of the invention are useful in the treatment and/or prevention of diseases or disorders mediated by T lymphocytes and/or PKC, e.g. acute or chronic rejection of organ or tissue allo- or xenografts or T-cell mediated inflammatory or autoimmune diseases, e.g. atherosclerosis, vascular occlusion due to vacular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock, or traumatic shock. The compounds of formula I are also useful in the treatment and/or prevention of T-cell mediated acute or chronic inflammatory diseases or disorders or autoimmune diseases e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis.

In accordance with the foregoing the present invention further provides:

2.1 A method for preventing or treating disorders or diseases mediated by T lymphocytes and/or PKC, e.g. such as indicated above, in a subject in need of such treatment, which method comprises administering to said subject an effective amount of a salt of the invention or a crystalline form thereof;

2.2 A method for preventing or treating acute or chronic transplant rejection or T-cell mediated inflammatory or autoimmune diseases, e.g. as indicated above, in a subject in need of such treatment, which method comprises administering to said subject an effective amount of a salt of the invention or a crystalline form thereof;

3. A salt of the invention or a crystalline form thereof, for use as a pharmaceutical, e.g. in any of the methods as indicated under 2.1 and 2.2 above.

4. A pharmaceutical composition, e.g. for use in any of the methods as in 2.1 and 2.2 above comprising a salt of the invention or a crystalline form thereof, in association with a pharmaceutically acceptable diluent or carrier therefor.

5. A salt of the invention or a crystalline form thereof, for use in the preparation of a pharmaceutical composition for use in any of the method as in 2.1 and 2.2 above.

6. A salt of the invention, or a crystalline form thereof, whenever prepared by a process as defined above.

Salt of the invention or a crystalline form thereof, e.g. mesylate salt or maleate salt of 3-(1.H.-indol-3-yl)-4-[2-(4-methyl-piperazin-1-yl)-quinazolin-4-yl]-pyrrole-2,5-dione may be administered as the sole active ingredient or together with other drugs in immunomodulating regimens or other anti-inflammatory agents e.g. for the treatment or prevention of allo- or xenograft acute or chronic rejection or inflammatory or autoimmune disorders. For example, they may be used in combination with cyclosporines, or ascomycines or their immunosuppressive analogs or derivatives, e.g. cyclosporin A, cyclosporin G, FK-506, ABT-281, ASM 981; an mTOR inhibitor, e.g. rapamycin, 40-O-(2-hydroxy)ethyl-rapamycin etc.; corticosteroids; cyclophosphamide; azathioprene; methotrexate; an accelerating lymphocyte homing agent, e.g. FTY 720; leflunomide or analogs thereof; mizoribine; mycophenolic acid; mycophenolate mofetil; 15-deoxyspergualine or analogs thereof; immunosuppressive monoclonal antibodies, e.g., monoclonal antibodies to leukocyte receptors, e.g., MHC, CD2, CD3, CD4, CD 11a/CD18, CD7, CD25, CD 27, B7, CD40, CD45, CD58, CD 137, ICOS, CD150 (SLAM), OX40, 4-1BB or their ligands, e.g. CD154; or other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4 lg (for ex. designated ATCC 68629) or a mutant thereof, e.g. LEA29Y, or other adhesion molecule inhibitors, e.g. mAbs or low molecular weight inhibitors including LFA-1 antagonists, Selectin antagonists and VLA-4 antagonists. Salt of the invention or a crystalline form thereof, e.g. mesylate salt or maleate salt of 3-(1.H.-indol-3-yl)-4-[2-(4-methyl-piperazin-1-yl)-quinazolin-4-yl]-pyrrole-2,5-dione, may also be administered together with an antiproliferative drug, e.g. a chemotherapeutic drug, e.g. in cancer treatment, or with an anti-diabetic drug in diabetes therapy.

In accordance with the foregoing the present invention provides in a yet further aspect:

7. A method as defined above comprising co-administration, e.g. concomitantly or in sequence, of a therapeutically effective amount of a salt of the invention or a crystalline form thereof, e.g. mesylate salt or maleate salt of 3-(1.H.-indol-3-yl)-4-[2-(4-methyl-piperazin-1-yl)-quinazolin-4-yl]-pyrrole-2,5-dione, and a second drug substance, said second drug substance being an immunosuppressant, immunomodulatory, anti-inflammatory, antiproliferative or anti-diabetic drug, e.g. as indicated above.

8. A therapeutic combination, e.g. a kit, comprising a) a salt of the invention or a crystalline form thereof, e.g. mesylate salt or maleate salt of 3-(1.H.-indol-3-yl)-4-[2-(4-methyl-piperazin-1-yl)-quinazolin-4-yl]-pyrrole-2,5-dione, and b) at least one second agent selected from an immunosuppressant, immunomodulatory, anti-inflammatory, antiproliferative and anti-diabetic drug. Component a) and component b) may be used concomitantly or in sequence. The kit may comprise instructions for its administration.

The salts of the present invention are synthesized in accordance with the following examples which are illustrative without limiting the scope of the present invention.

### Example 1

### Preparation of Mesylate Salt of 3-(1.H.-indol-3-yl)-4-[2-(4-methyl-piperazin-1-yl)-quinazolin-4-yl]-pyrrole-2,5-dione

2.63 g (6 mmol) of the free base of 3-(1.H.-indol-3-yl)-4-[2-(4-methyl-piperazin-1-yl)-quinazolin-4-yl]-pyrrole-2,5-dione and 40 ml of absolute ethanol are charged in a 250 ml 3-neck flask equipped with a mechanical stirrer, reflux condenser and an addition funnel. The mixture is heated to 45° C. and to the almost clear solution are added dropwise 0.39 ml of methanesulfonic acid (6 mol, 1 eq) diluted in 5 ml of absolute ethanol. The initially clear solution affords the precipitation of a solid and the mixture is kept at 45° C. for tow hours. It is subsequently cooled to room temperature and the yellow-orange solid is recovered by filtration. It is washed once by cold ethanol and dried overnight under vacuum.

### Example 2

### Preparation of Maleate Salt of 3-(1.H.-indol-3-yl)-4-[2-(4-methyl-piperazin-1-yl)-quinazolin-4-yl]-pyrrole-2,5-dione

2.63 g (6 mmol) of the free base of 3-(1.H.-indol-3-yl)-4-[2-(4-methyl-piperazin-1-yl)-quinazolin-4-yl]-pyrrole-2,5-dione and 40 ml of absolute ethanol are charged in a 250 ml 3-neck flask equipped with a mechanical stirrer, reflux condenser and an addition funnel. The mixture is heated to 45° C. and to the almost clear solution are added dropwise 0.70 g of maleic aid (6 mol, 1 eq) dissolved in 5 ml of absolute ethanol. Solid precipitation is almost immediate and the mixture is kept at 45° C. for two hours. It is subsequently cooled to room temperature and the orange solid is recovered by filtration. It is washed twice by cold 2-propanol and dried overnight under vacuum.

1. The free base of 3-(1.H.-indol-3-yl)-4-[2-(4-methyl-piperazin-1-yl)-quinazolin-4-yl]-pyrrole-2,5-dione and a salt thereof with an acid selected from the group consisting of benzoic, hydrochloric, citric, fumaric, lactic, maleic, malic, malonic, methanesulfonic, phosphoric, succinic, and tartaric acid.

2. A salt of 3-(1.H.-indol-3-yl)-4-[2-(4-methyl-piperazin-1-yl)-quinazolin-4-yl]-pyrrole-2,5-dione in crystalline or solvate form wherein the salt is formed with an acid selected from the group consisting of benzoic, hydrochloric, citric, fumaric, maleic, malic, malonic, methanesulfonic, succinic and tartaric acid.

3. A salt according to claim 1 or a crystalline form according to claim 2 wherein the acid is maleic or methanesulfonic.

4. A salt of 3-(1.H.-indol-3-yl)-4-[2-(4-methyl-piperazin-1-yl)-quinazolin-4-yl]-pyrrole-2,5-dione or crystalline form thereof according to claim 1 which is present in essentially pure form.

5. A process for the preparation of a salt of 3-(1.H.-indol-3-yl)-4-[2-(4-methyl-piperazin-1-yl)-quinazolin-4-yl]-pyrrole-2,5-dione according to claim 1 comprising reacting the free base of 3-(1.H.-indol-3-yl)-4-[2-(4-methyl-piperazin-1-yl)-quinazolin-4-yl]-pyrrole-2,5-dione with an acid, optionally in presence of a salt forming agent, and recovering from the reaction mixture the resultant salt, wherein the acid is selected from the group consisting of benzoic, hydrochloric, citric, fumaric, lactic, maleic, malic, malonic, methanesulfonic, phosphoric, succinic, and tartaric acid.

6. A process for the preparation of a benzoate, hydrochloric, citrate, fumarate, malate, malonate, mesylate, succinate, or tartarate salt of 3-(1.H.-indol-3-yl)-4-[2-(4-methyl-piperazin-1-yl)-quinazolin-4-yl]-pyrrole-2,5-dione in crystalline form according to claim 2 comprising appropriately converting the free base of 3-(1.H.-indol-3-yl)-4-[2-(4-methyl-piperazin-1-yl)-quinazolin-4-yl]-pyrrole-2,5-dione from a solution into crystalline salt thereof under crystallization-inducing conditions.

7. A pharmaceutical composition comprising a salt according to claim 1.

8. A pharmaceutical composition according to claim 7 wherein the salt is present in essentially pure form.

9. A method of treating diseases or disorders mediated by T lymphocytes and/or PKC, such as acute or chronic rejection of organ or tissue allo- or xenografts or T-cell mediated inflammatory or autoimmune diseases, comprising administering to a patient in need thereof a therapeutically effective amount of a salt according to claim 1.

10. A therapeutic combination comprising a) a salt according to claim 1 and b) at least one second agent selected from an immunosuppressant, immunomodulatory, anti-inflammatory, antiproliferative and anti-diabetic drug.
